(19) United States
(12) Patent Application Publication
(10) Pub. No.: US 2025/0143663 A1
(43) Pub. Date: May 8, 2025

(54) Medical Imaging System And Methods

(71) Applicant: Mobius Imaging, LLC, Shirley, MA (US)

(72) Inventor: Florian Herrmann, Schwanau (DE)

(73) Assignee: Mobius Imaging, LLC, Shirley, MA (US)

(21) Appl. No.: 18/836,814

(22) PCT Filed: Feb. 10, 2023
(86) PCT No.: PCT/US2023/012767

§ 371 Date: Aug. 8, 2024

Related U.S. Application Data
(60) Provisional application No. 63/308,648, filed on Feb. 10, 2022.

(51) Int. Cl. A61B 6/00; A61B 6/03; A61B 34/10; A61B 90/00

(52) U.S. Cl. CPC A61B 6/547; A61B 6/03; A61B 34/10; A61B 90/06; A61B 90/39; A61B 2090/3966

(57) ABSTRACT

The disclosure generally provides for a surgical system comprising an imaging device including a gantry supported for translation between a first position and a second position to obtain imaging data of a target site of a patient over a scan period, and a position sensor to generate gantry data representing a position of the gantry relative to the base between the first position and the second position; trackers including a gantry tracker operatively attached to the gantry; a navigation system including a localizer to track states of the trackers in a navigation reference frame; a controller operatively connected to the imaging device and the navigation system, and configured to register the imaging data to the navigation reference frame by synchronizing the gantry data generated by the position sensor with changes in tracked states of the gantry tracker occurring over the scan period representing movement between the first and second position.

### CROSS-REFERENCE TO RELATED APPLICATION

The subject patent application claims priority to and all the benefits of U.S. Provisional Patent Application No. 63/308,648, filed on Feb. 10, 2022, the disclosure of which is hereby incorporated by reference in its entirety.

### BACKGROUND

Conventional medical imaging devices, such as computed tomography (CT) and magnetic resonance (MR) imaging devices, are typically realized with fixed or otherwise relatively immobile devices located in a discrete area reserved for imaging that is often far removed from the point-of-care where the devices could be most useful.

Navigation systems are frequently utilized to assist medical professionals in carrying out various types of surgical procedures, including neurosurgical and orthopedic procedures. To this end, a surgeon may utilize a navigation system to track, monitor, or otherwise locate one or more tools, surgical instruments, and/or portions of a patient's anatomy within a common reference frame. Typically, tools and/or surgical instruments are tracked together with patient anatomy, and their relative movement is depicted on a display.

Conventional navigation systems may employ light signals, sound waves, magnetic fields, radio frequency signals, and the like, in order to track the position and/or orientation of objects. Often, trackers are attached or otherwise integrated into the object being tracked. A localizer cooperates with tracking elements (e.g., fiducials, markers, and the like) coupled to the tracker to monitor the tracker, and ultimately to determine a position and/or orientation of the object being tracked.

For certain procedures, patient-specific imaging data may be acquired intraoperatively using one or more types of imaging systems to help assist the surgeon in visualizing, navigating relative to, and/or treating the anatomy. To this end, navigation systems may cooperate with imaging systems and/or other parts of surgical systems (e.g., surgical tools, instruments, surgical robots, and the like) to track objects relative to a target site of the anatomy.

### SUMMARY

The disclosure generally provides for a surgical system comprising an imaging device, a plurality of trackers, a navigation system, and a controller. The imaging device includes a base, a gantry supported for translation relative to the base between a first gantry position and a second gantry position to obtain imaging data of a target site of a patient over a scan period, and a position sensor to generate gantry data representing a position of the gantry relative to the base between the first gantry position and the second gantry position. The plurality of trackers include a gantry tracker operatively attached to the gantry. The navigation system including a localizer to track states of the plurality of trackers in a navigation reference frame. The controller is operatively connected to the imaging device and to the navigation system. The controller is configured to register the imaging data to the navigation reference frame by synchronizing the gantry data generated by the position sensor with changes in tracked states of the gantry tracker occurring over the scan period representing movement of the gantry between the first gantry position and the second gantry position.

The disclosure further provides for a surgical system comprising an imaging device, a plurality of trackers, a navigation system, and a controller. The imaging device includes a base, a gantry supported for translation relative to the base between a first gantry position and a second gantry position to obtain imaging data of a target site of a patient over a scan period, and a position sensor to generate gantry data representing a position of the gantry relative to the base between the first gantry position and the second gantry position. The plurality of trackers includes a patient tracker adapted for attachment relative to the target site, and a gantry tracker operatively attached to the gantry. The navigation system includes a localizer to track states of the plurality of trackers in a navigation reference frame. The controller is operatively connected to the imaging device and to the navigation system and the controller is configured to register the imaging data to the navigation reference frame by synchronizing the gantry data generated by the position sensor with changes in tracked states of the gantry tracker occurring over the scan period representing movement of the gantry between the first gantry position and the second gantry position.

The disclosure further provides for a surgical system comprising an imaging device, a plurality of trackers, a navigation system, a plurality of inertial sensors, and a controller. The imaging device includes a base, and a gantry supported for translation relative to the base between a first gantry position and a second gantry position to obtain imaging data of a target site of a patient over a scan period. The gantry supports an x-ray source and an x-ray detector for rotation about an axis. The plurality of trackers includes a gantry tracker operatively attached to the gantry. The navigation system includes a localizer to track states of the plurality of trackers in a navigation reference frame. The plurality of inertial sensors includes an imaging inertial sensor operatively attached to one of the x-ray source and the x-ray detector to generate imaging inertial data. The controller is operatively connected to the imaging device, the navigation system, and the plurality of inertial sensors. The controller is configured to register the imaging data to the navigation reference frame by synchronizing the imaging inertial data generated by the imaging inertial sensor with changes in tracked states of the gantry tracker occurring over the scan period representing movement of the gantry between the first gantry position and the second gantry position.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of an imaging system.

FIG. 2 is a cross-sectional schematic illustration of an imaging system that illustrates the rotating and non-rotating portions of the system.

FIG. 3 is a perspective view of the imaging system of FIG. 1 with the outer shell of the gantry rendered transparent without the patient support.

FIGS. 4A-4B are side views of a mobile imaging system with a drive wheel and casters extended and the base of the system raised off the floor.

FIG. 5 is a bottom isometric view of the imaging system showing the drive wheel and casters.

FIGS. 6A-6B are perspective schematic views of the imaging system in a first position and in a second position.

FIG. 7 illustrates an imaging system performing a helical scanning procedure between moving from the first position towards the second position.

FIG. 8 is a schematic view of an inertial measurement unit.

FIG. 9 depicts the imaging system with the inertial measurement units in communication with the localizer, the x-ray source, and the x-ray detector.

FIG. 10 illustrates an example of an ideal scan trajectory and an actual trajectory of an image scan when an error is present.

FIG. 11 illustrates a schematic view of one example of a rotational scan path of the x-ray source and detector on the rotor.

FIG. 12 illustrates a helical path and a scan trajectory.

FIG. 13 illustrates one example of the imaging system including a fiducial plate on the side of the patient support.

### DETAILED DESCRIPTION

The various versions of the present disclosure will be described in detail with reference to the accompanying drawings. Wherever possible, the same reference numbers will be used throughout the drawings to refer to the same or corresponding parts. References made to particular examples and implementations are for illustrative purposes and are not intended to limit the scope of the present disclosure.

The present disclosure generally relates to a medical imaging system 100 (also known as a surgical imaging system). The medical imaging system 100 may be used for pre-operative planning, intraoperative use, and/or post-operative follow up. The medical imaging system 100 may function with an x-ray imaging device 10 (and/or other types of imaging devices) to acquire x-ray images (e.g., patient imaging data) of one or more anatomical objects of interest and display the x-ray images to a surgeon or surgery team. For example, the medical imaging system 100 may take and display an x-ray image of a particular patient P anatomical feature or region (e.g., knee, spine, ankle, foot, neck, hip, arm, leg, rib cage, hand, shoulder, head, the like, and/or combinations thereof). In some examples, the medical imaging system 100 may function to superimpose an image of surgical instruments 106, 108 over the displayed x-ray image of the anatomical feature, displaying the surgical instruments 106, 108 relative the anatomical feature. The medical imaging system 100 may function to acquire multiple x-ray images forming a CT scan of a patient P. The medical imaging system 100 may be configured to automatically correlate a position of an x-ray imaging device 10 with a portion of the x-ray images taken during a scan. The medical imaging system 100 may register the x-ray images with the position of the x-ray images based on information generated by one or more of an optical sensor (e.g., camera units 56 of a localizer 54), gravity vector sensors 57, 67, 77, 87, or both. In some versions, the medical imaging system 100 comprises an x-ray imaging device 10 (also referred to as an imager) including a mobile base 20, a gimbal 30, a gantry 40, and a pedestal 50. The gantry 40 is configured to translate along the base 20. The medical imaging system 100 also may comprise a navigation system 16 including a localizer 54, a plurality of tracking devices 58, 132, 134, 136 (also referred to as “trackers”) to be tracked or otherwise monitored by the navigation system 16, and one or more controllers 17, 113 operatively connected with the x-ray imaging device 10, the navigation system 16, and the plurality of tracking devices 58, 132, 134, 136. The navigation system 16 may monitor the position and/or orientation of one or more tracking devices 57, 67, 77, 87 located on the x-ray source 43, the x-ray detector 45 detector, the localizer 54, the base 20, or combinations thereof.

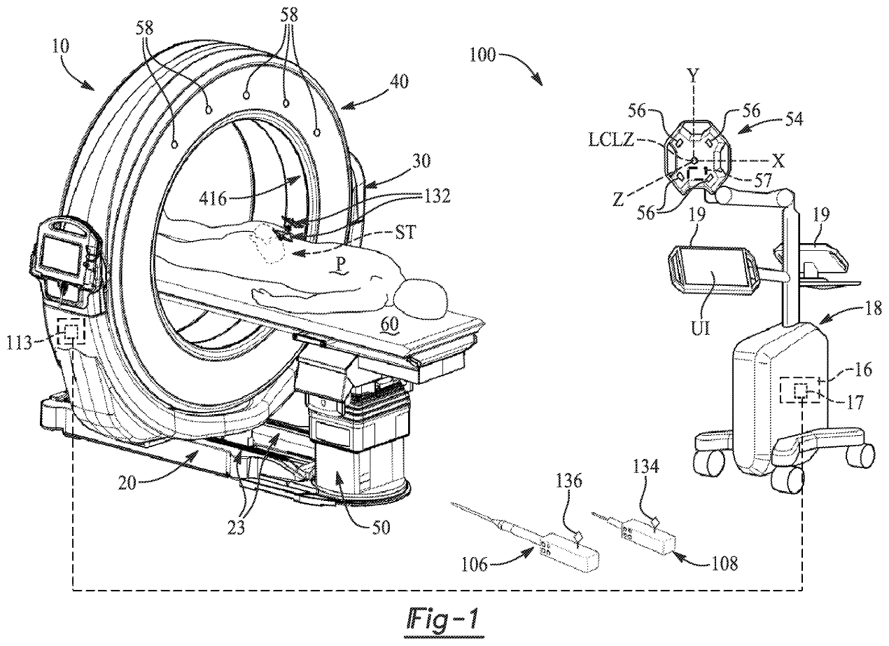

Referring to FIGS. 1-5, in some versions, the navigation system 16 may employ a navigation controller 17 that communicates with an imager system controller 113 of the x-ray imaging device 10. The medical imaging system 100 is configured to collect imaging data, such as, for example x-ray computed tomography (CT) or magnetic resonance imaging (MRI) data, from an object located within a bore 416 of the gantry 40, in any manner known in the medical imaging field, and to register the collected imaging data in a navigation reference frame of the navigation system 16.

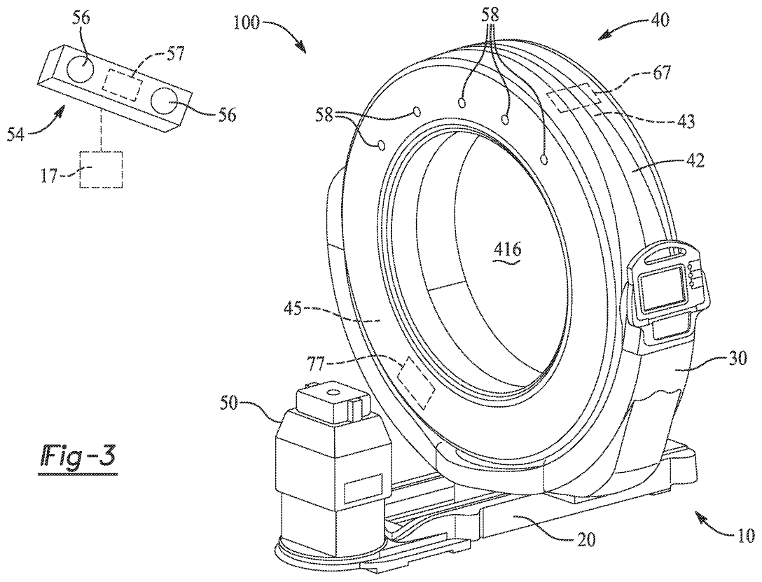

As shown in FIGS. 1-4B, the pedestal 50 is adapted to support a tabletop support 60 that can be attached to the pedestal 50 in a cantilevered manner and extend out into the bore 416 of the gantry 40 to support a patient P or other object being imaged. As shown in FIG. 3, the tabletop support 60 can be partially or entirely removed from the pedestal 50, and the gantry 40 can be rotated relative to the base 20, preferably at least about 90 degrees, from an imaging position (FIGS. 1 and 2-5) to a transport position to facilitate transport and/or storage of the x-ray imaging device 10.

The x-ray imaging device 10 functions to acquire images of the patient P or anatomical features of the patient's P body supported on the tabletop support 60 (or on some other type of patient support). The x-ray imaging device 10 may include a structure with an emitting portion realized as an x-ray source 43 (e.g., one or more x-ray tubes or other types of radiation sources) and an imaging portion realized as an x-ray detector 34 (or some other form of detector). The x-ray imaging device 10 may be configured to have a generally O-shape gantry 40. The O-shaped gantry may include the x-ray source 43 and the x-ray detector 45 located on the opposing portions of the gantry 40. The x-ray source 43 and the x-ray detector 45 may be at a fixed distance from each other. An imaging region (not shown in detail) may be defined in the center of the O-shape, within the bore 416, between the x-ray source 43 and the x-ray detector 45. A patient P or a portion of a patient P may be located in the center of the bore 416 of the gantry 40, between the x-ray source 43 and the x-ray detector 45, so that a specific portion of the patient P may be imaged.

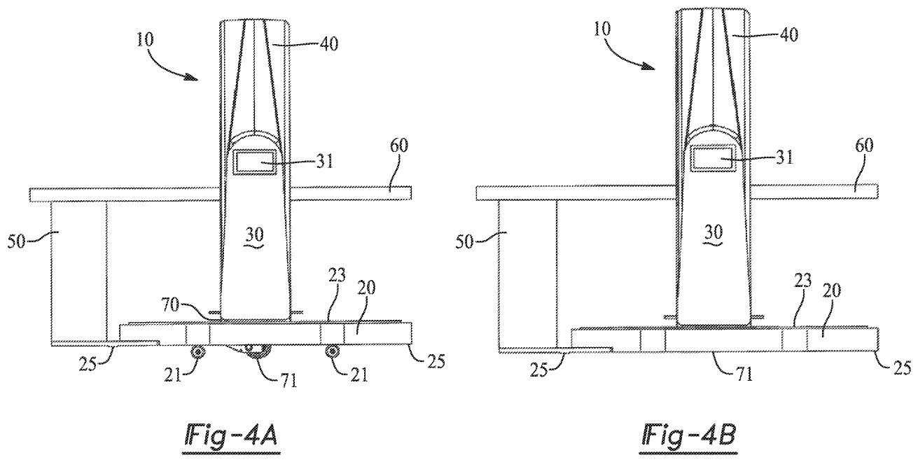

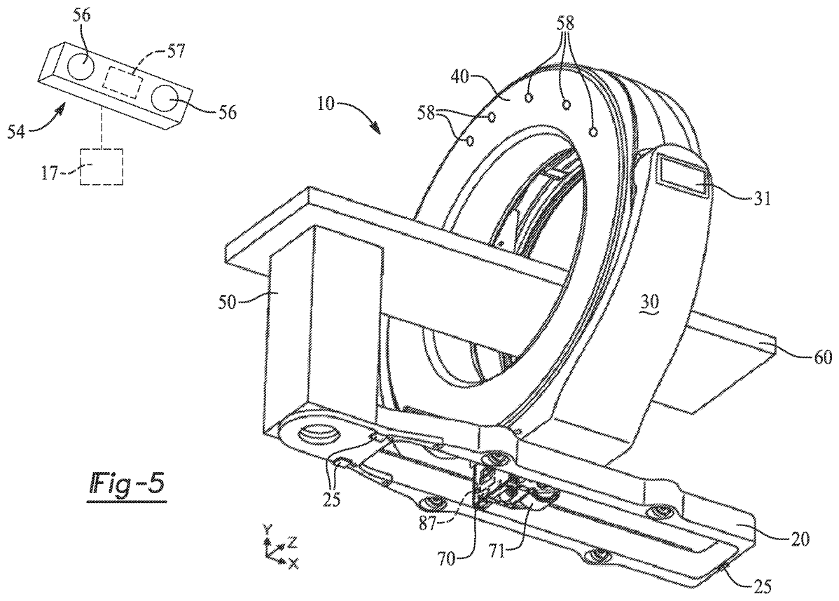

As is best depicted in FIGS. 4A-5, the x-ray imaging device 10 includes a drive mechanism 70 mounted beneath the gimbal 30 and the gantry 40 and within the base 20. The drive mechanism 70 also comprises a drive wheel 71 that can extend and retract between a first extended position (see FIG. 4A) to facilitate transport of the x-ray imaging device 10, and a second retracted position (see FIG. 4B) during an image acquisition procedure (e.g., during an imaging scan). The drive mechanism 70 includes a main drive (not shown in detail) that is geared into the drive wheel 71 when the drive wheel 71 is in the first extended position (FIG. 4A) to propel the x-ray imaging device 10 across a floor or other surface, and thus facilitate transport and positioning of the x-ray imaging device 10. In some versions, the drive wheel 71 can be decoupled from the main drive when the drive wheel 71 is in the second retracted position, thus preventing the x-ray imaging device 10 from back-driving the main drive during an imaging procedure. In some versions, the drive mechanism 70 includes one or more sensors (not shown) to track the position of the drive wheel 7, the position of the gimbal 30 and gantry 40, and the like, relative to the base 20 and/or to other components of the x-ray imaging device 10.

As is illustrated in FIGS. 3-4B, the base 20 is realized as a sturdy, generally rectilinear support structure, and includes a central opening extending lengthwise along the base 20 in which the drive mechanism 70 is positioned. As seen in FIG. 5, the bottom of the base 20 includes a plurality of pockets (not shown in detail) that contain retractable casters 21. The casters 21 can be spring-loaded and biased to extend from the bottom of the base 20 when the x-ray imaging device 10 is raised off the ground, as shown in FIG. 4A. When the drive wheel 71 is retracted and the x-ray imaging device 10 is lowered to the ground, as shown in FIG. 4B, the casters 21 are retracted into their respective pockets. In an alternative version, an active drive system, rather than a passive spring-based system, can drive the extension and retraction of the casters in their respective pockets.

The gimbal 30 may be a generally C-shaped support that is mounted to the top surface of base 20 and includes a pair of arms 31, 33 extending up from the base. The arms 31, 33 may be connected to opposite sides of gantry 40 so that the gantry is suspended above base 20 and gimbal 30. In some versions, the gimbal 30 and gantry 40 may rotate together about a first (e.g., vertical) axis with respect to the base 20, and the gantry 40 may tilt about a second (e.g., horizontal) axis with respect to the gimbal 30 and base 20. In some versions, a gimbal drive mechanism (not shown in detail) may be mounted between the gimbal 30 and the base 20 to controllably drive the rotation (i.e., “yaw” motion) of the gimbal 30 and gantry 40 with respect to the base 20. A gimbal drive mechanism may also controllably drive the “tilt” motion of the gantry 40 with respect to the gimbal 30.

The gimbal 30 and gantry 40 may translate with respect to the base 20. The gimbal 30 may include bearing surfaces (not shown in detail) that travel on rails 23, as shown in FIG. 1, to provide the translation motion of the gimbal 30 and gantry 40. A scan drive mechanism (not shown in detail) may drive the translation of the gantry 40 and gimbal 30 relative to the base 20, and a main drive mechanism may drive the entire system in a transport mode (e.g., on one or more casters or wheels). In the version of FIG. 1, both of these functions are combined in the drive mechanism 70 that is located beneath the gimbal 30. Further details of similar drive mechanisms 70 for x-ray imaging devices 10 are described in U.S. Pat. No. 8,753,009, filed Feb. 11, 2011, the entire disclosure of which is hereby incorporated by reference.

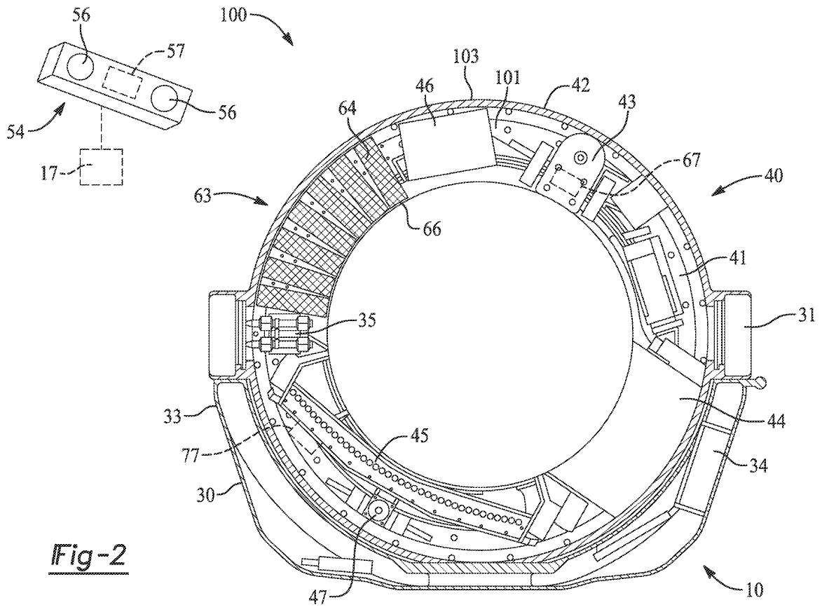

The version illustrated in FIG. 2 illustrates a single high voltage generator 44 powering the x-ray source 43. However, it will be understood that in various versions multiple high voltage generators 44 may be provided on the gantry 40, and each x-ray source 43 may have a dedicated high voltage generator 44. In some versions, one or more high voltage generators 44 may be provided off of the gantry 40, and high voltage power may be delivered to the x-ray source 43 via a cable or slip ring system (not shown).

The x-ray imaging device 10 generally operates to obtain images of an object located in the bore 416 of the gantry 40. For example, in the case of an x-ray CT scan, the rotor 41 rotates within the housing of the gantry 40 while imaging components, including the x-ray source 43 and x-ray detector 45, obtain image data at a variety of scan angles. Generally, the x-ray imaging device 10 obtains image data over relatively short intervals, with a typical scan lasting less than a minute, or sometimes just a few seconds. During these short intervals, however, a number of components, such as the x-ray source 43 and the high-voltage generator 44, require a large amount of power, including, in some versions, up to 32 kW of power.

The high voltage generator 44 may be powered by a power source on the gantry 40, such as a battery system 63. As shown in FIG. 2, the battery system 63 may be mounted to and rotates with the rotor 41. The battery system 63 may include a plurality of electrochemical cells. The cells may be incorporated into one or more battery packs. The battery system 63 is preferably rechargeable and may be recharged by a charging system (not shown) between imaging operations, such as when the rotor 41 is not rotating. In some versions, the battery system 63 consists of lithium iron phosphate (LiFePO4) cells, though it will be understood that other suitable types of batteries can be utilized.

The battery system 63 provides power to various components of the x-ray imaging device 10. In particular, since the battery system 63 is located on the rotor 41, the battery system 63 may provide power to any component on the rotor 41, even as these components are rotating with respect to the non-rotating portion of the x-ray imaging device 10. Specifically, the battery system 63 is configured to provide the voltages and peak power required by the high-voltage generator 44 and x-ray source 43 (e.g., the x-ray tube) to perform an imaging scan. For example, a battery system 63 may output ˜360 V or more, which may be stepped up to 120 kV at the high-voltage generator 44 to perform an imaging scan. In addition, the battery system 63 may provide power to operate other components, such as an on-board computer 46, the x-ray detector arrays 45, and a drive mechanism 47 for rotating the rotor 41 within the gantry 40. Here, in some versions, the drive mechanism 47 drives the rotation of the rotor 41 around the interior of the gantry 40. The drive mechanism 47 may be controlled by an imager system controller 113 that controls the rotation and precise angular position of the rotor 41 with respect to the gantry 40, such as by using position feedback data from one or more encoder devices (not shown). The drive mechanism 47 may include a motor and gear system mounted to the rotor 41 (see FIG. 2; not shown in detail). The motor may drive a gear that may be engage with a mating component on the non-rotating portion of the x-ray imaging device 10 to drive the rotation of the rotor 41. For example, a belt 82 may be rotatably fixed on the non-rotating portion of the x-ray imaging device 10 (e.g., the outer shell of the gantry 40), such as on a circumferential rail. The drive mechanism 47 may engage with the belt 82 to drive the rotation of the rotor 41 within the gantry 40. The drive mechanism 47 may be powered by the battery system 63, may be secured to the rotor 41, and may be positioned behind the x-ray detector 45, as shown in FIG. 2. Further details of a similar type of drive mechanisms 47 are described in U.S. Pat. No. 9,737,273, filed Apr. 6, 2012, the entire disclosure of which is hereby incorporated by reference.

An on-board computer 46 may be provided on the rotating portion of the system and may be secured to rotor 41 in a suitable location, as shown in FIG. 2. The on-board computer 46 may be powered by battery system 63. The on-board computer 46 may be any suitable computing device, and may include one or more processors having associated memory that may execute instructions (e.g., software) stored in memory, as is known in the art. The on-board computer 46 may perform various control functions for the various components on the rotor 41 and may serve as an interface between components on the rotor 41 and other components of the x-ray imaging device 10. The computer 46 may be configured to receive imaging data collected by the x-ray detector 45. For example, the x-ray detector 45 may stream their image data over a suitable data connection (e.g., wired or wireless) to the on-board computer 46. The computer 46 may store, process and/or transmit the imaging data. For example, the on-board computer 46 may include or may be coupled to a wireless transmitter that may transmit the data to another logical entity, such as to an external workstation and/or to another computer located on the non-rotating portion of the system (e.g., in the gimbal 30). This may enable real-time display of the collected imaging data.

A docking system 35 may be provided for connecting the rotating portion of the x-ray imaging device 10 to the non-rotating portion between imaging scans. The docking system 35 may include a connector for carrying power between the rotating and non-rotating portions. In some versions, the docking system 35 may be used to provide power to the battery system 63 such that the batteries may be charged using power from an external power source (e.g., grid power). The docking system 35 may also include a data connection to allow data signals to pass between the rotating and non-rotating portions. Further details of a suitable docking system are described in U.S. Pat. No. 9,737,273, filed Apr. 6, 2012, the entire disclosure of which is hereby incorporated by reference.

FIG. 3 is a perspective view of the medical imaging system 100, and shows the x-ray imaging device 10 with the outer shell 42 of the gantry 40 depicted as being transparent for illustrative purposes. In some versions, the medical imaging system 100 may provide mobile bi-plane imaging, such as x-ray fluoroscopic imaging. The rotor 41 may rotate to any angle to obtain images in any desired imaging plane. The x-ray source 43 and detector 45 may be mounted to the rigid, circular rotor 41 so as to inhibit relative movement of the imaging components during a scan, such as to prevent flexing towards or away from the bore 416. Further, the entire rotor 41 assembly may be housed within a rigid outer shell 42 of the gantry 40, as shown in FIG. 3, which may further constrain the rotor 41 and prevent relative movement of the imaging components.

The medical imaging system 100 may be used to perform arterial “road mapping” imaging, according to some versions. It is often the case that a physician would like to get a picture of the arterial anatomy over all or a portion of a patient's P body. What is typically done is a contrast agent is injected at a first location in the patient P, and an imaging device (such as an x-ray fluoroscopic C-arm device) is manually moved to various locations along the patient's P body to capture images as the contrast agent works its way through the body and into the patient's P extremities. The various images may be combined to provide a fuller image (or roadmap) of the patient's P arterial anatomy. The medical imaging system 100 of the present disclosure may be used to provide an arterial roadmap (e.g., a single plane roadmap, or a bi-plane roadmap). A contrast agent may be injected into a patient P positioned within the bore 416 of the system. The gantry 40 and gimbal 30 may be driven along rails 23 on the base 20 to obtain arterial images as the contrast agent works its way through the patient P. The movement of the gantry 40 and gimbal 30 on the rails 23 may be controlled by an operator or may be controlled automatically by a pre-programmed road mapping tracking controller, which may track the flow of contrast agent within the region of interest of the patient P based on a known or likely flow path of the contrast agent over time. In some versions, an image analysis of the flow of contrast agent in one or more arteries of the patient P may be used to automatically determine the velocity of the gantry 40 (e.g., how quickly the gantry should translate down the patient axis), as the gantry 40 translates on the base 20.

The medical imaging system 100 may further be used to perform cone beam CT imaging. The rotor 41 may rotate within the gantry 40 while the x-ray detector 45 obtain images. The image data may then be reconstructed using a tomographic algorithm as is known in the art to obtain a 3D reconstructed image of the object. In some examples, the x-ray detector 45 may obtain images which may be combined for the reconstruction. Thus, in some versions, the rotor 41 may only need to rotate a portion of the distance that would normally be required (e.g., a 90° rotation of the rotor 41 may enable the detector to scan 180° of the object, a 270° rotation of the rotor 41 enables a full 360° scan of the object). In some versions, the gantry 40 and gimbal 30 may be translated along rails 23 during cone beam CT imaging to provide a helical cone beam CT scan (FIG. 5). In some versions, a helical cone beam scan may be coordinated with the injection of a contrast agent to provide a three-dimensional arterial roadmap image.

As described above, the high-voltage generator 44 may provide high-voltage power to the x-ray source 43. In some versions, the high-voltage generator 44 may generate a pulsed power signal to one or more radiation source for fluoroscopy applications, and may generate continuous power to one or more radiation sources for CT scanning.

As noted above, the x-ray imaging device 10 includes the x-ray source 43, such as an x-ray tube, that is configured to direct radiation, including collimated x-ray radiation, onto the x-ray detector 45. The x-ray source 43 may include a beam steering mechanism that may alter the direction of the output beam by a particular angle, such as 90° or more. In some examples, the x-ray imaging device may include two or more radiation sources and two or more detectors such that at least a portion of the output radiation beam is alternately centered on a first detector and a second detector, which may be spaced by 90° to provide bi-planar imaging, such as described in U.S. Pat. No. 9,526,461, filed Jun. 25, 2013, the entire disclosure of which is hereby incorporated by reference.

Various versions of the x-ray imaging device 10 may be configured so as to be relatively compact. Various components may be designed to fit efficiently within the housing of the gantry 40. For example, the high voltage generator 44 may have one or more angled or curved surfaces to accommodate the curvature of the rotor 41 and/or the gantry 40. The battery system 63 may also include angled or curved surfaces to accommodate the curvature of the rotor 41 and/or gantry 40. In some versions, the outer shell 42 of the gantry 40 may comprise both a protective outer covering for the rotating portion and a mounting surface for a bearing that enables the rotating portion 101 to rotate 360° within the outer shell 42 of the gantry 40 while, at the same time, affording a relatively low-profile enclosure of the components of the gantry 40.

The outer diameter of the gantry 40 can be relatively small, which may facilitate the portability of the x-ray imaging device 10. In a preferred version, the outer diameter of the gantry 40 is less than about 70 inches, such as between about 60 and 68 inches, and in some versions is about 66 inches. The outer circumferential wall of the outer shell 42 may be relatively thin to minimize the outer diameter dimension of the gantry 40. In addition, the interior diameter of the gantry 40, or equivalently the bore 416 diameter, can be sufficiently large to allow for the widest variety of imaging applications, including enabling different patient supports 60 (e.g., tabletop supports 60) to fit inside the bore 416, and to maximize access to a subject located inside the bore 416. In some versions, the bore diameter of the gantry 40 is greater than about 38 inches, such as between about 38 and 44 inches, and in some versions can be between about 40 and 50 inches. In one exemplary version, the bore 416 has a diameter of about 42 inches. The gantry 40 generally has a narrow profile, which may facilitate portability of the x-ray imaging device 10. In some versions, the width of the gantry 40 is less than about 17 inches and can be about 15 inches or less.

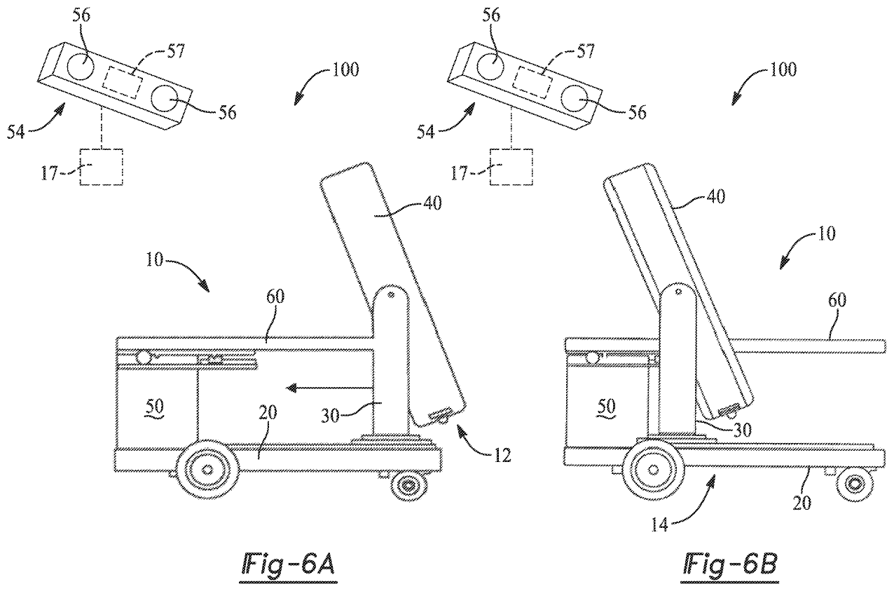

As mentioned above, the gantry 40 may be moved between a plurality of positions, and is configured to translate and/or tilt about the base 20 of the x-ray imaging device 10. The gantry 40 is configured to move relative the base 20 to capture x-ray images of a patient P or anatomical feature of interest (e.g., a target site ST), at one or more angled relative to a patient P or particular anatomical feature, raise, lower, repositioned, or a combination thereof. During movement, the x-ray source 43 and the x-ray detector 45 maintain a fixed relationship, keeping the same distance on the opposite ends of the gantry 40. As best seen in FIGS. 4A and 4B, the gantry 40 is configured to move between a first position 12 and second position 14 and may include a plurality of intermediate positions (e.g., transistor and/or intermittent movement) between the first position 12 and the second position 14. FIG. 6A illustrates the gantry 40 in a first position 12 and FIG. 6B illustrates the gantry 40 moved to the second position 14 along the base 20.

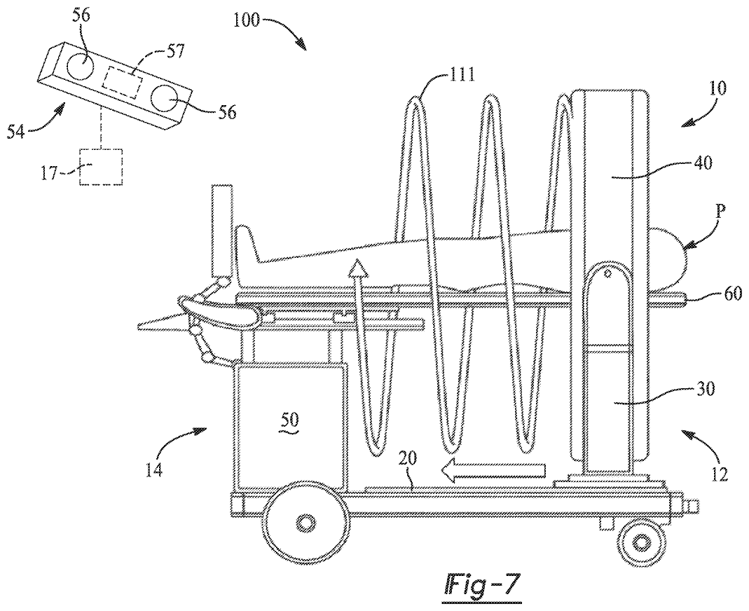

As the x-ray imaging device 10 is moved between the first position 12 and the second position 14, the x-ray imaging device 10 may perform a scan. Moving the gantry 40 between the first position 12 and the second position 14 while the x-ray imaging device 10 takes x-ray images defines a scan period. As the x-ray imaging device 10 generates imaging data when performing a scan. In some examples, the x-ray imaging device 10 performs a scout scan where the x-ray source 43 and the x-ray detector 45 remain rotationally fixed during the scan while the gantry 40 is translated between the first position 12 and the second position 14. FIG. 7 illustrates a helical scanning imaging application of the present disclosure. The imaging equipment (e.g., the x-ray source 43 and the x-ray detector 45) rotate around the interior of the gantry 40 to obtain imaging data, while the gantry 40 and gimbal 30 simultaneously translate along the base 20. The arrow 111 in FIG. 7 indicates the path of the imaging equipment around the patient P in a helical scan. The x-ray imaging device 10 is thus able to obtain true helical scan x-ray CT images.

Referring back to FIG. 1, as noted above, the medical imaging system 100 includes the navigation system 16. One example of the navigation system 16 is described in U.S. Pat. No. 9,008,757, filed on Sep. 24, 2013, the entire disclosure of which is hereby incorporated by reference. The navigation system 16 tracks movement of various objects, such as, for example, portions of the x-ray imaging device 10 (e.g., gantry 40, rotor 41, base 20, pedestal 50, tabletop support 60), one or more surgical instruments 106, 108 or tools, anatomy of a patient P (e.g., the spine or other bone structures, such as one or more vertebra, the pelvis, scapula, or humerus), and/or combinations thereof. The navigation system 16 monitors or otherwise tracks these objects, and may gather state information of each object with respect to a (navigation) localizer coordinate system LCLZ. As used herein, the state of an object includes, but is not limited to, data that defines the position and/or orientation of the tracked object (e.g., coordinate systems thereof) or equivalents/derivatives of the position and/or orientation. For example, the state may be a pose of the object, and/or may include linear velocity data, angular velocity data, and the like. In some examples, such as shown in FIG. 1, the navigation controller 17 is operatively connected with the imager system controller 113.

The navigation system 16 may employ a mobile cart assembly 18 that houses a navigation controller 17, and/or other types of control units. A navigation user interface UI is in operative communication with the navigation controller 17. The navigation user interface UI includes one or more display devices 19. The navigation system 16 is capable of displaying graphical representations of the relative states of the tracked objects to the user using the one or more display devices 19. The navigation user interface UI further comprises one or more input devices (not shown in detail) to input information into the navigation controller 17 or otherwise to select/control certain aspects of the navigation controller 17. Such input devices include interactive touchscreen displays. However, the input devices may include any one or more of push buttons, pointer, foot switches, a keyboard, a mouse, a microphone (voice-activation), gesture control devices, and the like. In some examples, the user may use buttons located on the pointer 106 to navigate through icons and menus of the user interfaces UI to make selections, configuring the medical imaging system 100 and/or advancing through the workflow.

In the illustrated versions, the localizer 54 of the navigation system 16 is coupled to the navigation controller 17. In some versions, the localizer 54 is an optical localizer and includes a camera unit 56. In certain configurations, the localizer 54 may be similar to as is described in U.S. Pat. No. 10,959,783 filed Apr. 15, 2016, the entire disclosure of which is hereby incorporated by reference. The localizer 54 may function to monitor and track tracking devices 58, 132, 134, 136 (also referred to as “trackers”) that are coupled to or otherwise supported on various tracked objects, such as the x-ray imaging device 10, surgical instruments 106, 108, the patient P, and/or combinations thereof. One suitable localizer 54 is the FP8000 tracking camera manufactured by Stryker Corporation (Kalamazoo, Mich.).

The localizer 54 may function to send information regarding the position of the tracking devices 58, 132, 134, 138 to the navigation controller 17. As noted above, the localizer 54 may include one or more camera units 56 (or other types of tracking sensors), which may function in the visible light spectrum, the infrared spectrum, or both. The one or more camera units 56 may utilize one or more optical sensing methods, such as CCD, CMOS, optical image, or a combination thereof. In some versions, magnetic sensing of detection may be used to acknowledge and track differences in magnetic fields. In some versions, radio frequency methods may function to track radio frequencies. In some examples, the localizer 54 may be positioned in any location which has a line of sight to trackers 136, 134 on the surgical instruments 106, 108, to one or more patient trackers 132, and/or combinations thereof. In some examples, the localizer 54 may incorporate one or more additional forms of sensing such as radio frequency, magnetic, or both, in addition to or in place of the camera units 56. The localizer 54 may be in a fixed position, or may be movable. For example, the localizer 54 may be attached to a stationary, or may be located on a moveable base such as the mobile cart assembly 18.

As mentioned above, the navigation system 16 includes or otherwise cooperates with a plurality of tracking devices 58, 132, 134, 136 (also referred herein as “trackers”) configured to be tracked by the navigation system 16. The tracking devices 58, 132, 134, 136 may function to be sensed and tracked by the camera units 56 of the localizer 54. The trackers 58, 132, 134, 136 may be realized in various ways, and may include different features and/or structure adapted to be sensed by the navigation system 16. The one or more trackers 58, 132, 134, 136 may include LEDs, reflective surfaces, patterns, magnetic coils, radio transmitters, and/or optically identifiable geometric shapes that uniquely defines position and orientation perceivable by the navigation system 16. The trackers 58, 132, 134, 136 may be optical, magnetic, radio frequency, or a combination thereof. In some examples, tracking the gantry 40 with the navigation system 16 may be required in order to determine the position and orientation relative to the localizer coordinate system LCLZ (also known as the navigation reference frame). To this end, one or more gantry trackers 58 may be realized with active or passive markers that can be attached to the inner, front or backside surfaces of the gantry 40. In the example shown in FIG. 1, the gantry trackers 58 are configured as optical trackers and are attached to the gantry 40. In some versions, one or more patient trackers 132 may be used to track anatomy of the patient P, and one or more tool trackers 134, 136 may be coupled to surgical instruments such as a pointer 106 or a rotary instrument 108. The trackers 58, 132, 134, 136 can be registered to their respective objects (e.g., the gantry 40, the patient P, and the instruments 106, 108) and the navigation system 16 manually, automatically, or a combination thereof.

As noted above, the navigation system 16 may track the position and/or movement of one or more surgical instruments 106, 108 configured to probe, cut, saw, drill, grind, debride, cauterize, probe, or otherwise manipulate or treat tissue of the patient P. The one or more surgical instruments 106, 108 may be realized by or on an end effector of a robotic arm (not shown). The surgical instruments 106, 108 may be handheld, robotic, powered, nonpowered, or combinations thereof. The surgical instruments 106, 108 may include more than one respective tracker 134, 136, or a single tracker 134, 146, that can be registered with the localizer 54 so that the localizer 54 may track the position of the surgical instruments 106, 108 in real time. The surgical instruments 106, 108 may be tracked and displayed superimposed in real time over the images acquired by the x-ray imaging device 10 so that the location of working ends of the surgical instruments 106, 108 relative the anatomy of the patient P is known.

The navigation system 16 may include one or more patient trackers 132 adapted for attachment to the anatomy of the patient P. The patient trackers 132 may function with the navigation system 16 to detect and compensate for movement and deformations during a procedure, such as to allow a surgeon and/or surgery team to know the real-time poses of the surgical instruments 106, 108 relative to the target site ST of the anatomy of the patient P. The patient tracker 132 may include at least one tracking element (e.g., a fiducial, a reflective marker, an LED, and the like). The patient tracker 132 may be any shape that is suitable to track movement of a patient P during a procedure. The patient tracker 132 is not limited to a particular shape or form, and may be rigid, flexible, and/or have multiple separate sections. In one example, the patient tracker 132 has a plurality of tracking elements, such as LEDs, disposed on a flexible substrate having the shape of a generally rectangular frame with an open window there through that can be removably secured to the patient's P skin with adhesive.

Turning to FIG. 2, the navigation system 16 includes a plurality of position sensors 57, 67, 77, 87. In some examples, the position sensors are gravity vector sensors 57, 67, 77, 87 (also referred to as inertial measurement units (IMUs)). The gravity vector sensors 57, 67, 77, 87 may function to detect the direction toward center of Earth gravity, regardless of position or orientation. In some versions, the direction towards the center of Earth gravity is a gravity vector. The gravity vector sensors 57, 67, 77, 87 determine the gravity vector regardless of the position of the gravity vector sensor 57, 67, 77, 87. The gravity vector may be used as a reference point to determine the position of another object, such as gravity vector sensor 57 located with the localizer 54, since the gravity vector sensors 57, 67, 77, 87 provides the gravity vector which is always toward the center of Earth gravity. In some examples, the gravity vector sensor 57 is associated with the localizer 54 to serve as a reference point, since the localizer 54 is typically stationary during a scanning procedure. In this example, the gravity vector may be referenced to determine the angular position of one or more elements of the x-ray imaging device 10 during a scan by tracking and analyzing the positional information generated by position sensors 67, 77, 87 associated with portions of the x-ray imaging device 10 comparing the relative position of the position sensors 67, 77, 87 of the x-ray imaging device 10 with gravity vector sensor 57 of the localizer. In FIG. 2, position sensors 67, 77 are configured as gravity vector sensors and are located on the x-ray source 43 and the x-ray detector 45. The gravity vector sensors 57, 67, 77, 87 may be located in various locations so long as a connection with the navigation system 16 can be maintained. For example, the gravity vector sensors 57, 67, 77, 87 may be located within the localizer 54, the x-ray source 43 of the x-ray imaging device 10, the x-ray detector 45 of the x-ray imaging device 10, the rotor 41 of the x-ray imaging device 10, the base 20 of the x-ray imaging device 10, or combinations thereof. The gravity vector sensors 57, 67, 77, 87 may be connected with the navigation system 16 through wired connections, wireless connections, a network, or combinations thereof. As is best depicted in FIGS. 2-3, one position sensor 57 is configured as a gravity vector sensor 57 and is located with the localizer 54, while other position sensors 67, 77 are configured as gravity vector sensors 67, 77 located on the x-ray source 43 and on the x-ray detector 45.

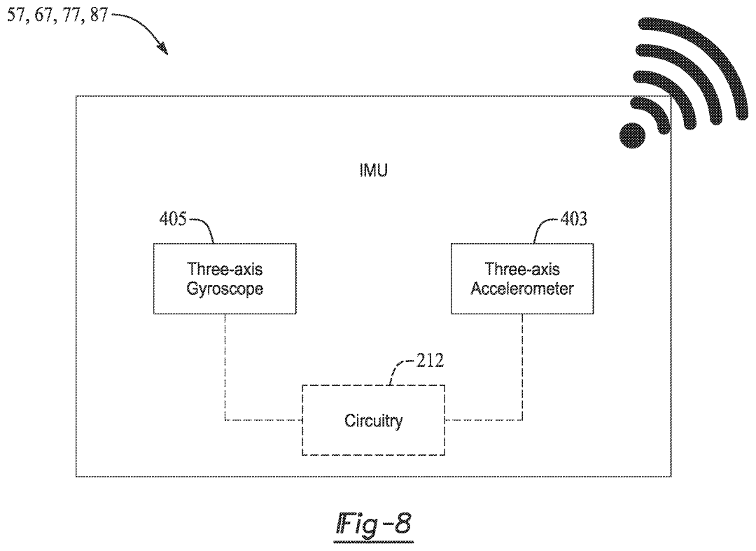

One or more of the gravity vector sensors 57, 67, 77, 87 may be realized as inertial measurement units (IMU) 57, 67, 77, 87 as noted above, and may be used to register elements of the medical imaging system 100 to one another. In some examples, such as shown in FIG. 8, each of the inertial measurement units 57, 67, 77, 87 may include a three-axis accelerometer 403 and a three-axis gyroscope 405. The accelerometer 403 and gyroscope 405 may be fabricated utilizing Micro-Electro-Mechanical (MEMS) technology. The accelerometer 403 and the gyroscope 405 may be separate components (e.g., chips) located in the corresponding device or structure that the gravity vector sensor 57, 67, 77, 87 is connected with, or may be integrated on with the corresponding device or structure (e.g., integrated circuit).

The localizer 54, the x-ray source 43, the x-ray detector 45, or combinations thereof may include circuitry 212 coupled to the accelerometer 403 and gyroscope 405, forming the gravity vector sensors 57, 67, 77, 87, that may be configured to read output signals from these components 403, 405. The accelerometer 403 may output signals measuring the linear acceleration of each of the gravity vector sensors 57, 67, 77, 87 connected with the localizer 54, the x-ray source 43, the x-ray detector 45, respectively, such as in three-dimensional space. The gyroscope 405 may output signals measuring the angular velocity of the gravity vector sensors 57, 67, 77, 87, such as in three-dimensional space. The signals from the accelerometer 403 and gyroscope 405 may be processed using a suitable processor, such as an imager system controller 113, the navigation controller 17, or both, to determine the position and orientation of each of the gravity vector sensors 57, 67, 77, 87 with respect to an initial inertial reference frame via a dead reckoning technique. Here, integrating the angular velocity measurements from the gyroscope 405 enables the current orientation of the gravity vector sensors 57, 67, 77, 87 to be determined with respect to a known starting orientation. Similarly, integrating the linear acceleration measurements from the accelerometer 403 enables the current velocity of the gravity vector sensors 57, 67, 77, 87 to be determined with respect to a known starting velocity. A further integration enables the current position of the gravity vector sensors 57, 67, 77, 87 to be determined with respect to a known starting position. Each of the gravity vector sensors 57, 67, 77 are set on a synchronized clock. In some examples, an additional gravity vector sensor 87 is located on the drive mechanism 70, and translates with the gantry 40 between the first position 12 and second position 14 (shown in FIG. 5). This gravity vector sensor 87 functions the same as the other gravity vector sensors 57, 67, 77 described herein.

In some versions, measurement data from the gravity vector sensors 57, 67, 77, 87 may transmitted from the localizer 54, the drive mechanism 70, the x-ray source 43, and/or the x-ray detector 45, and/or to a separate computing device (e.g., navigation controller 17 and/or imaging system controller) via a wired or wireless link. The data may be transmitted wirelessly using a suitable wireless communication protocol or standard (e.g., an IEEE 802.15x (BLUETOOTH®) or IEEE 802.11 (Wi-Fi) connection), as described above. The navigation controller 17 may perform the inertial navigation calculations to determine the position and orientation of the gravity vector sensors 57, 67, 77, 87 in three-dimensional space, such as within the localizer coordinate system LCLZ or some other common coordinate system (and/or may translate between different coordinate systems). The inertial navigation calculations may be initialized with a known initial position, orientation, and/or velocity data associated with the localizer 54, the drive mechanism 70, the x-ray source 43, and/or the x-ray detector 45, which may be known or otherwise derived from the most recent tracking data received from the navigation system 16.

In some versions, at least a portion of the inertial navigation calculations may be performed on the localizer 54, the drive mechanism 70, the x-ray source 43, and/or the x-ray detector 45, such as on a processor (e.g., microprocessor) located therewithin or, in some examples, with the on-board computer 46. In some versions, the navigation controller 17, the imager system controller 113, or both, may perform at least a portion of the inertial navigation calculations. Here, inertial navigation may be initialized using motion tracking data from one or more external sources (e.g., the localizer 54), which may be received by the gravity vector sensor 87 on the drive mechanism 70, the gravity vector sensor 67 on the x-ray source 43, and/or the gravity vector sensor 77 on the x-ray detector 45, over a wired or wireless link.

In some versions, inertial position tracking may be performed in parallel with motion tracking using the camera units 56 of the localizer 54. In some versions, optical tracking data and inertial navigation data may be fused in the navigation system 16, registering the scan data with the navigation reference frame (e.g., relative to the localizer coordinate system LCLZ). For example, the plurality of gantry trackers 58, and the position sensors 67, 77 may be configured as gravity vector sensors that are connected with the x-ray imaging device 10, and may be used to monitor the position of the x-ray imaging device 10 when the x-ray imaging device 10 is acquiring scan data while moving from the first position 12 to the second position 14. The gantry trackers 58 and the gravity vector sensors 67, 77 are tracked by the navigation system 16 to monitor the area which the x-ray imaging device 10 has scanned. By tracking the x-ray imaging device 10, the image data can be registered with the navigation reference frame LCLZ.

When tracking the x-ray source 43 and the x-ray detector 45 by inertial navigation, the accuracy of the tracking may be acceptable over a particular time frame, which may be known or determined empirically. In certain applications, inertial navigation may subject to drift which may accumulate over time to produce tracking accuracy errors that can increase as a function of time. Thus, in some circumstances, after a pre-determined time period, inertial navigation data may not be sufficiently accurate to support continued tracking of the x-ray source 43 and the x-ray detector 45 absent a position state update using data from another source (e.g., the camera units 56 of the localizer 54). In some versions, the navigation system 16 may be configured to determine whether the inertial navigation data satisfies one or more navigation accuracy criteria for tracking the position and/or orientation of the x-ray source 43 and the x-ray detector 45. In some examples, the navigation accuracy criteria may include a time limit for tracking using only inertial navigation. The medical imaging system 100 may notify the user (e.g., via an audible and/or visual alert) in response to determining that the navigation accuracy criteria is not satisfied. The notification to the user may be provided on the display screen of a display devices 19.

As described above, multiple gravity vector sensors 57, 67, 77, 87, with each unit including a three-axis accelerometer 403 and a three-axis gyroscope 405, may be located on or within the localizer 54, the drive mechanism 70, the x-ray source 43, and the x-ray detector 45. In some versions, inertial navigation of the x-ray source 43 and the x-ray detector 45 may be performed based on an average of the results from each respective unit. This may enable accurate inertial navigation over a longer time period than when using a single inertial measurement unit. The medical imaging system 100 may notify the user (e.g., via an audible and/or visual alert) in response to determining that the inertial navigation is no longer considered accurate, which may be after pre-determined time period and/or when a variance in the calculated position and/or orientation of the instrument from a plurality of inertial measurement units IMUs exceeds a threshold value.

Because the mobile cart assembly 18 and the gantry 40 of the x-ray imaging device 10 can be positioned relative to each other and also relative to the patient P in the representative version illustrated in FIG. 1, the navigation system 16 can transform the coordinates of each tracker 58, 132, 134, 136 from the localizer coordinate system LCLZ into other coordinate systems (e.g., defined by different trackers 58, 132, 134, 136, localizers 54, and the like), or vice versa, so that navigation relative to the target site ST (or control of surgical instruments 106, 108) can be based at least partially on the relative positions and orientations of multiple trackers 58, 132, 134, 136 within a common coordinate system (e.g., the localizer coordinate system LCLZ). In some examples, the navigation controller 17 may calculate the pose of the gantry trackers 58 to register the pose of the gantry 40 during the image scan between the first position 12 and the second position 14 based on monitoring the gantry trackers 58. Furthermore, the navigation system 16 may utilize gravity vector sensors 57, 67, 77, 87 to assist in registration of the components of the imaging system with the localizer coordinate system LCLZ. Other configurations are contemplated.

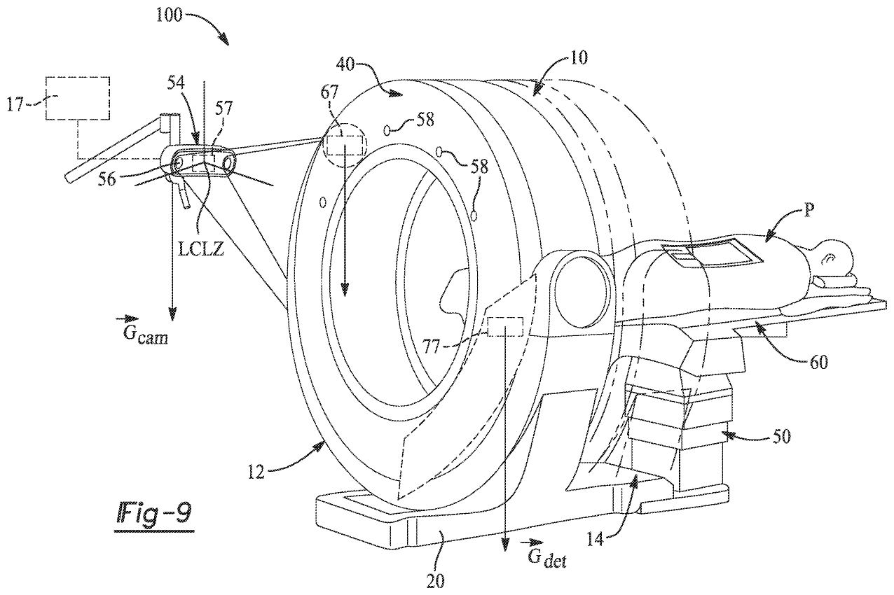

Turning to FIGS. 6A-7 and 9, the navigation controller 17 can analyze information received from the localizer 54 and the gravity vector sensors 57, 67, 77, 87, and can determine the pose of one or more portions of the x-ray imaging device 10 (e.g. x-ray source 43; x-ray detector 45) while the x-ray imaging device 10 acquires images during a scan. The navigation controller 17 may receive data from the localizer 54 and the gravity vector sensors 57, 67, 77, 87 used to calculate a scan vector using the gravity vector sensor 57 within the localizer 54 as a reference. The scan vector comprises angular position data of the gravity vector sensors 67, 77 placed on the x-ray source 43 and x-ray detector 45, respectively, of the x-ray imaging device 10 acquired during the scan between the first position 12 and the second position 14 relative to the gravity vector sensor 57 of the localizer 54. During a scan procedure, once the gantry 40 is positioned for the scan at the first position 12, a precise measurement of the gravity vector is taken from the gravity vector sensors 67, 77 on the x-ray imaging device 10 relative to the gravity vector sensor 57 on the localizer 54. During the scan, as the gantry 40 is translated along the base 20 between the first position 12 and the second position 14, multiple measurements are taken of the angular velocity by gravity vector sensors 67, 77 on the x-ray imaging device 10 and are analyzed with the gravity vector measurement of gravity vector sensor 57 on the localizer 54. With the information of the gravity vector sensors 67, 77 on the x-ray imaging device, and their relation to the gravity vector sensor 57 on the localizer 54, a normalized relationship can be determined.

In some examples, the x-ray source 43 and x-ray detector 45 are rotated during a helical scan, and are rotationally stationary during a scout scan. In either example, the angular position data generated by the gravity vector sensors 67, 77 is monitored and analyzed by the navigation controller 17 to register the acquired images of the scan with the navigation reference frame.

As described above, the navigation controller 17 may further calculate the sensed position of the gantry trackers 58 between the first position 12 of the gantry 40 and a second position 14 of the gantry 40 which is used in conjunction to the angular position data of the gravity vector sensors 57, 67, 77 to register the image scan data to the navigation reference frame. In some other examples, the navigation controller 17 may calculate the sensed positions of the gantry trackers 58 to register the position of the gantry 40 at one or more locations during the image scan as the x-ray imaging device 10 is moved between the first position 12 and the second position 14 by generating a scan vector based on the gantry trackers 58 on the x-ray imaging device 10 and gravity vector data from the gravity vector sensor 57 located on the localizer 54, the gravity vector sensor 67 located on the x-ray source 43, the gravity vector sensor 77 located on the x-ray detector 45, and the gravity vector sensor 87 located on the drive system 70. The navigation controller 17 may calculate a determined angle which is the angular difference between the perceived location of the gantry trackers 58 by the localizer 54 and the gravity vector of each of the gravity vector sensors 57, 67, 77, 87. In one example, such as during a scan, the gantry trackers 58 on the gantry 40 are optically tracked by the localizer 54 and the gravity vector sensor 57 on the localizer 54 measures the direction of the gravitational vector of the gravity vector sensors 67, 77, 87 in communication with the x-ray source 43 and the x-ray detector 45. Once the gantry 40 is positioned for the scan at the first position 12, the localizer 54 captures the position of the gantry trackers 58, and a precise measurement of the gravity vector is taken from the gravity vector sensors 67, 77, 87 relative to the gravity vector sensor 57. During the scan, as the gantry 40 is translated along the base 20 and the tabletop support 60 between the first position 12 and the second position 14, multiple measurements are taken of the angular velocity by gravity vector sensors 67, 77, 87, and analyzed with the gravity vector measurement of gravity vector sensor 57 on the localizer 54. The gravity vector sensor 67 located on the x-ray source 43 and the gravity vector sensor 77 located on the x-ray detector 45 are used to monitor angular movement, rotational movement, and/or linear movement of the x-ray source 43 and x-ray detector 45 during the scan between the first position 12 and the second position 14. The gravity vector sensor 87 located on the drive system 70 may be used to monitor linear movement of the drive system 70 which carries the gimbal 30 and the gantry 40. With the information of the gravity vector sensors 67, 77, 87 and their relation to the gravity vector sensor 57, a normalized relationship is known. Further, the localizer 54 may monitor the position of the gantry trackers 58 at one or more locations and/or time intervals during the scan.

In some examples, the localizer 54 may capture the position and orientation of the gantry trackers 58 at the first position 12. In other examples, the localizer 54 may capture the position and orientation of the gantry trackers 58 at the first position 12 and the second position 14. In another example, the localizer 54 may capture the position and orientation of gantry trackers 58 at a plurality of intermittent positions between the first position 12 and the second position 14. By capturing the position and orientation of the gantry trackers 58 on the gantry 40, positional data of the x-ray imaging device 10 is created and can be updated or verified by the gravity vector data obtained by the relationship of gravity vector sensors 67, 77, 87 relative to gravity vector sensor 57 throughout the scan between the first position 12 and the second position 14.

In some examples, the gravity vector sensor 67 on the x-ray source 43 and the gravity vector sensor 77 on the x-ray detector 45 may generate rotational data of the scan, which the navigation system 16 analyzes relative to the reference gravity vector sensor 57 on the localizer 54. By comparing the rotational scan data, the navigation system 16 may determine intermittent or interrupted rotor 41 rotation. The navigation controller 17 and the imager system controller 113 work in conjunction to register the image scan data with the navigation reference frame. Additional position sensors, such as a gravity vector sensor 87 on the drive mechanism 70, may also be used to for further accuracy of registering the image scan data to the navigation reference frame.

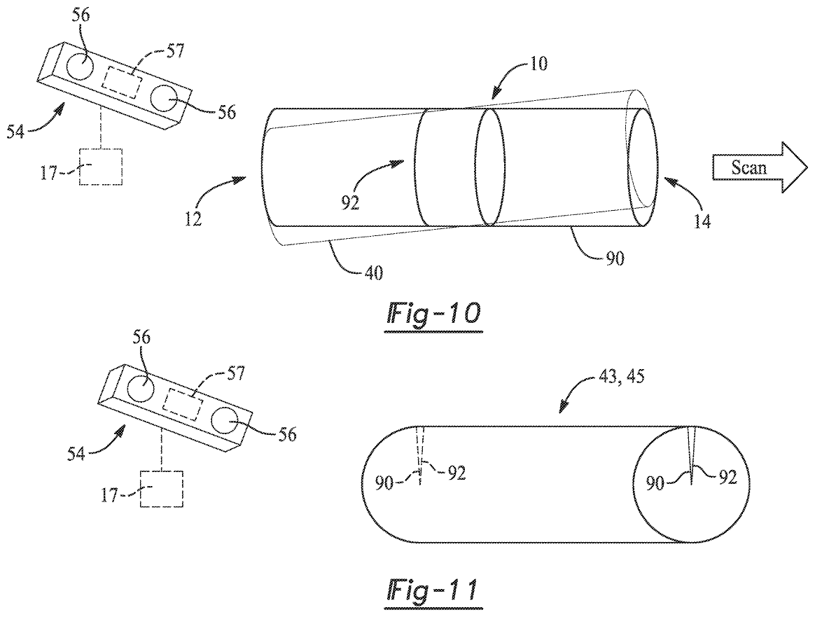

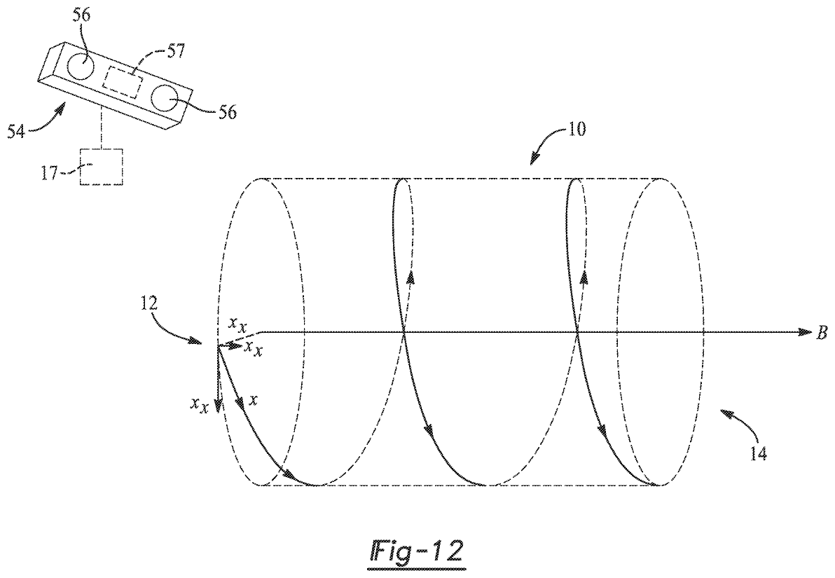

By tracking the gantry trackers 58 with the camera units 56 of the localizer 54 and/or analyzing the difference in angular position of the gravity vector sensor 57 relative to the gravity vector sensors 67, 77 located on the x-ray source 43 and x-ray detector 45, registration of the image data generated by the x-ray imaging device 10 to the navigation reference frame LCLZ is afforded with significant accuracy, being based on one or more sets of reference location data. For example, turning to FIG. 10, without verification (e.g., monitoring the gantry trackers 58 at one or more locations; analyzing gravity vector sensors 57, 67, 77, 87), modeled movement 90 of the movement of the gantry 40 is time synced during the scan to register the image scan data with the navigation coordinate system. However, in this example, if the gantry 40 is tilted or yawed, such as illustrated via an actual scan path 92, the registration of the image scan data with the navigation reference frame will be inaccurate. Similarly, in FIG. 11, during a rotational scan, rotating the x-ray source 43 and the x-ray detector 45, the actual scan path 92 (e.g., path/rotation) may not correspond with the modeled movement 90 (e.g., path/rotation 90), which may lead to an inaccurate registration of image scan data. Conversely, when the methods of monitoring the position and orientation of the gantry 40, the x-ray source 43, and/or x-ray detector 45 are utilized, the accuracy of the position and orientation of the scan image data registered with the navigation reference frame becomes more accurate, such as is illustrated by FIG. 12.

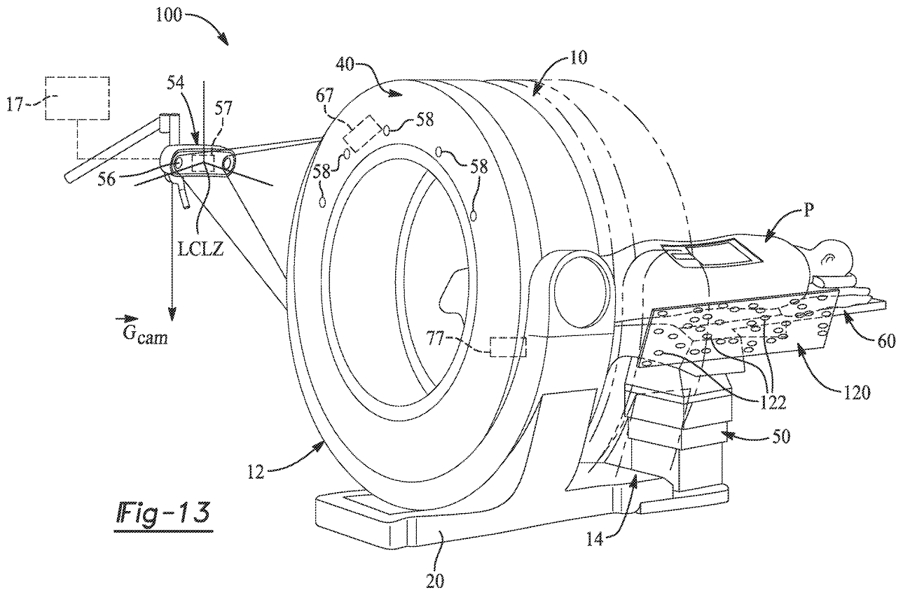

FIG. 13 illustrate another example of the imaging system 100 including a fiducial plate 120. The fiducial plate 120 includes a plurality of radio opaque fiducial markers 122 that appear in image scan data. The fiducial markers 122 are arranged with a specific geometry such that, during a scan, the identification of the specific geometry arrangement enables the imager system controller 113 and navigation controller 17 to readily identify the location of the scan and register the image data with the navigation reference frame.

The navigation controller 17 may further process positional data of the trackers 134, 136 on surgical instruments 106, 108, the patient trackers 132 or both. For example, the navigation system 16 may display the position of the surgical instrument(s) 106, 108 onto a display device 19, showing the position of the one or more surgical instruments 106, 108 relative to an anatomical feature (e.g., target site ST) as the surgical instrument 106, 108 is moved during the procedure.

The each of the computer processors, such as the imager system controller 113, navigation controller 17, and the on-board computer 46 each include a memory. The memory may function to hold one or more libraries, databases, lookup tables, or a combination thereof. The memory may function to store data relating to the positions of the x-ray imaging device 10 (scan vector), the images taken by the x-ray imaging device 10 during a scan, the gravity vector value, a plurality of image identifiers corresponding to the position of the trackers 58, the gravity vector sensors 57, 67, 77, 87, or a combination thereof. The memory may be transitory memory, non-transitory memory, or both.

In this application, including the definitions below, the term “controller” may be replaced with the term “circuit.” The term “controller” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The one or more controller(s) may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The one or more controllers may communicate with other controllers using the interface circuit(s). Although the controller may be depicted in the present disclosure as logically communicating directly with other controllers, in various configurations the controller may actually communicate via a communications system. The communications system includes physical and/or virtual networking equipment such as hubs, switches, routers, and gateways. In some configurations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various configurations, the functionality of the controller may be distributed among multiple controllers that are connected via the communications system. For example, multiple controllers may implement the same functionality distributed by a load balancing system. In a further example, the functionality of the controller may be split between a server (also known as remote, or cloud) controller and a client (or, user) controller.

Some or all hardware features of a controller may be defined using a language for hardware description, such as IEEE Standard 1364-2005 (commonly called “Verilog”) and IEEE Standard 10182-2008 (commonly called “VHDL”). The hardware description language may be used to manufacture and/or program a hardware circuit. In some configurations, some or all features of a controller may be defined by a language, such as IEEE 1666-2005 (commonly called “SystemC”), that encompasses both code, as described below, and hardware description.

The various controller programs may be stored on a memory circuit. The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SENSORLINK, and Python®.

Several examples have been discussed in the foregoing description. However, the examples discussed herein are not intended to be exhaustive or limit the disclosure to any particular form. The terminology that has been used is intended to be in the nature of words of description rather than of limitation. Many modifications and variations are possible in light of the above disclosure and the disclosure may be practiced otherwise than as specifically described.

The present disclosure also comprises the following clauses, with specific features laid out in dependent clauses, that may specifically be implemented as described in greater detail with reference to the configurations and drawings above.

### CLAUSES

I. A surgical system comprising:
an imaging device including:
   a base,
   a gantry supported for translation relative to the base between a first gantry position and a second gantry position to obtain imaging data of a target site of a patient over a scan period, and
   a position sensor to generate gantry data representing a position of the gantry relative to the base between the first gantry position and the second gantry position;
a plurality of trackers including a gantry tracker operatively attached to the gantry;
navigation system including a localizer to track states of the plurality of trackers in a navigation reference frame; and
a controller operatively connected to the imaging device and to the navigation system, the controller being configured to register the imaging data to the navigation reference frame by synchronizing the gantry data generated by the position sensor with changes in tracked states of the gantry tracker occurring over a scan period representing movement of the gantry between the first gantry position and the second gantry position.

II. The surgical system of clause I, wherein the gantry includes an x-ray source and an x-ray detector supported for rotational movement about an axis.

III. The surgical system of clause II, further comprising an imaging inertial sensor in communication with the controller and operatively attached to one of the x-ray source and the x-ray detector to generate imaging inertial data; and
wherein the controller is further configured to register the imaging data to the navigation reference frame by synchronizing the imaging inertial data generated by the imaging inertial sensor with changes in tracked states of the gantry tracker occurring over the scan period representing movement of the gantry between the first gantry position and the second gantry position.

IV. The surgical system of clause III, further comprising a second imaging inertial sensor in communication with the controller and operatively attached to the other of the x-ray source and the x-ray detector to generate second imaging inertial data.

V. The surgical system of clause IV, wherein the controller is further configured to determine rotational acceleration of the gantry based on one or more of the imaging inertial data and the second imaging inertial data.

VI. The surgical system of any of clauses IV-V, wherein the controller is further configured to determine translational movement of the gantry based on one or more of the imaging inertial data and the second imaging inertial data.

VII. The surgical system of any of clauses III-VI, further comprising a navigation inertial sensor in communication with the controller and operatively attached to the localizer to generate reference inertial data; and
wherein the controller is further configured to normalize the imaging inertial data based on the reference inertial data.

VIII. The surgical system of clause VII, wherein at least one of the imaging inertial sensor and the navigation inertial sensor is configured as a gravity-vector sensor.

IX. The surgical system of any of clauses II-IX, wherein the gantry further includes a frame, and a rotor supported by the frame for rotation about the axis, with the x-ray source and the x-ray detector operatively attached to the rotor.

X. The surgical system of clause IX, wherein the x-ray source and the x-ray detector remain stationary relative to the frame while obtaining scout imaging data.

XI. The surgical system of any of clauses IX-X, wherein rotation of the rotor about the axis moves the x-ray source and the x-ray detector relative to the frame while obtaining three-dimensional imaging data.

XII. The surgical system of any of clauses I-XI, wherein the localizer of the navigation system is configured to optically track states of the plurality of trackers.

XIII. The surgical system of clause XII, wherein the localizer of the navigation system monitors tracked states of the gantry tracker occurring over the scan period based on optical movement of the gantry tracker occurring during initial movement of the gantry from the first gantry position, during movement interruption of the gantry at the second gantry position, and during intermittent movement of the gantry between the first gantry position and the second gantry position.

XIV. The surgical system of any of clauses I-XIII, wherein the imaging device further includes a motor to translate the gantry relative to the base.

XV. The surgical system of any of clauses I-XIV, wherein the plurality of trackers further includes a patient tracker adapted for attachment relative to the target site.

XVI. The surgical system of any of clauses I-XV, further comprising a tabletop support and a fiducial plate connected with the tabletop support, the fiducial plate including a plurality of radio opaque markers arranged in a specific geometry arrangement, the plurality of radio opaque markers configured to appear in the imaging data with the specific geometry arrangement;
wherein the controller is further configured to synchronize the specific geometry arrangement of the plurality of radio opaque markers in the imaging data with changes in tracked states of the gantry tracker occurring over the scan period representing movement of the gantry between the first gantry position and the second gantry position and the gantry data generated by the position sensor.

XVII. A surgical system comprising:
an imaging device including:
   a base,
   a gantry supported for translation relative to the base between a first gantry position and a second gantry position to obtain imaging data of a target site of a patient over a scan period, and
   a position sensor to generate gantry data representing a position of the gantry relative to the base between the first gantry position and the second gantry position;
a plurality of trackers a patient tracker adapted for attachment relative to the target site, and a gantry tracker operatively attached to the gantry;
a navigation system including a localizer to track states of the plurality of trackers in a navigation reference frame; and
a controller operatively connected to the imaging device and to the navigation system, the controller being configured to register the imaging data to the navigation reference frame by synchronizing the gantry data generated by the position sensor with changes in tracked states of the gantry tracker occurring over the scan period representing movement of the gantry between the first gantry position and the second gantry position.

XVIII. A surgical system comprising:
an imaging device including:
   a base,
   a gantry supported for translation relative to the base between a first gantry position and a second gantry position to obtain imaging data of a target site of a patient over a scan period, the gantry supporting an x-ray source and an x-ray detector for rotation about an axis;
a plurality of trackers including a gantry tracker operatively attached to the gantry;
a navigation system including a localizer to track states of the plurality of trackers in a navigation reference frame;
a plurality of inertial sensors including an imaging inertial sensor operatively attached to one of the x-ray source and the x-ray detector to generate imaging inertial data; and
a controller operatively connected to the imaging device, to the navigation system, and to the plurality of inertial sensors, the controller being configured to register the imaging data to the navigation reference frame by synchronizing the imaging inertial data generated by the imaging inertial sensor with changes in tracked states of the gantry tracker occurring over the scan period representing movement of the gantry between the first gantry position and the second gantry position.

XIX. The surgical system of clause XVIII, further comprising a second imaging inertial sensor in communication with the controller and operatively attached to the other of the x-ray source and the x-ray detector to generate second imaging inertial data.

XX. The surgical system of clause XIX, wherein the controller is further configured to determine rotational acceleration of the gantry based on one or more of the imaging inertial data and the second imaging inertial data.

XXI. The surgical system of any of clauses XIX-XX, wherein the controller is further configured to determine translational movement of the gantry based on one or more of the imaging inertial data and the second imaging inertial data.

XXII. The surgical system of any of clauses XVIII-XXI, further comprising a navigation inertial sensor in communication with the controller and operatively attached to the localizer to generate reference inertial data; and
wherein the controller is further configured to normalize the imaging inertial data based on the reference inertial data.

XXIII. The surgical system of clause XXII, wherein at least one of the imaging inertial sensor and the navigation inertial sensor is configured as a gravity-vector sensor.

XXIV. The surgical system of any of clauses XVIII-XXIII, wherein the gantry further includes a frame, and a rotor supported by the frame for rotation about the axis, with the x-ray source and the x-ray detector operatively attached to the rotor.

XXV. The surgical system of clause XXIV, wherein the x-ray source and the x-ray detector remain stationary relative to the frame while obtaining scout imaging data.

XXVI. The surgical system of any of clauses XXIV-XXV, wherein rotation of the rotor about the axis moves the x-ray source and the x-ray detector relative to the frame while obtaining three-dimensional imaging data.

XXVII. The surgical system of any of clauses XVIII-XXVI, wherein the localizer of the navigation system is configured to optically track states of the plurality of trackers.

XXVIII. The surgical system of clause XXVII, wherein the localizer of the navigation system monitors tracked states of the gantry tracker occurring over the scan period based on optical movement of the gantry tracker occurring during initial movement of the gantry from the first gantry position, during movement interruption of the gantry at the second gantry position, and during intermittent movement of the gantry between the first gantry position and the second gantry position.

XXIX. The surgical system of any of clauses XVIII-XXVIII, further comprising a tabletop support and a fiducial plate connected with the tabletop support, the fiducial plate including a plurality of radio opaque markers arranged in a specific geometry arrangement, the plurality of radio opaque markers configured to appear in the imaging data with the specific geometry arrangement;

wherein the controller is further configured to synchronize the specific geometry arrangement of the plurality of radio opaque markers in the imaging data with changes in tracked states of the gantry tracker occurring over the scan period representing movement of the gantry between the first gantry position and the second gantry position and the inertial data generated by the inertial sensors.

XXX. A surgical system comprising:
an imaging device including:
   a base,
   a tabletop support connected with the base and configured to support a patient;
   a gantry supported for translation relative to the base and the tabletop support between a first gantry position and a second gantry position to obtain imaging data of a target site of a patient over a scan period, and
   a position sensor to generate gantry data representing a position of the gantry relative to the base and the tabletop support between the first gantry position and the second gantry position;
a plurality of trackers including a gantry tracker operatively attached to the gantry;
a fiducial plate connected with the tabletop support, the fiducial plate including a plurality of radio opaque markers arranged in a specific geometry arrangement, the plurality of radio opaque markers configured to appear in the imaging data with the specific geometry arrangement;
a navigation system including a localizer to track states of the plurality of trackers in a navigation reference frame; and
a controller operatively connected to the imaging device and to the navigation system, the controller being configured to register the imaging data to the navigation reference frame by synchronizing the specific geometry arrangement of the plurality of radio opaque markers in the imaging data with changes in tracked states of the gantry tracker occurring over a scan period representing movement of the gantry between the first gantry position and the second gantry position and the gantry data generated by the position sensor.

1. A surgical system comprising:
an imaging device including:
a base,
a gantry supported for translation relative to the base between a first gantry position and a second gantry position to obtain imaging data of a target site of a patient over a scan period, and
a position sensor to generate gantry data representing a position of the gantry relative to the base between the first gantry position and the second gantry position;
a plurality of trackers including a gantry tracker operatively attached to the gantry;
a navigation system including a localizer to track states of the plurality of trackers in a navigation reference frame; and
a controller operatively connected to the imaging device and to the navigation system, the controller being configured to register the imaging data to the navigation reference frame by synchronizing the gantry data generated by the position sensor with changes in tracked states of the gantry tracker occurring over a scan period representing movement of the gantry between the first gantry position and the second gantry position.

2. The surgical system of claim 1, wherein the gantry includes an x-ray source and an x-ray detector supported for rotational movement about an axis.

3. The surgical system of claim 2, further comprising an imaging inertial sensor in communication with the controller and operatively attached to one of the x-ray source and the x-ray detector to generate imaging inertial data; and
wherein the controller is further configured to register the imaging data to the navigation reference frame by synchronizing the imaging inertial data generated by the imaging inertial sensor with changes in tracked states of the gantry tracker occurring over the scan period representing movement of the gantry between the first gantry position and the second gantry position.

4. The surgical system of claim 3, further comprising a second imaging inertial sensor in communication with the controller and operatively attached to the other of the x-ray source and the x-ray detector to generate second imaging inertial data.

5. The surgical system of claim 4, wherein the controller is further configured to determine rotational acceleration of the gantry based on one or more of the imaging inertial data and the second imaging inertial data.

6. The surgical system of claim 4, wherein the controller is further configured to determine translational movement of the gantry based on one or more of the imaging inertial data and the second imaging inertial data.

7. The surgical system of claim 3, further comprising a navigation inertial sensor in communication with the controller and operatively attached to the localizer to generate reference inertial data; and
wherein the controller is further configured to normalize the imaging inertial data based on the reference inertial data.

8. The surgical system of claim 7, wherein at least one of the imaging inertial sensor and the navigation inertial sensor is configured as a gravity-vector sensor.

9. The surgical system of claim 2, wherein the gantry further includes a frame, and a rotor supported by the frame for rotation about the axis, with the x-ray source and the x-ray detector operatively attached to the rotor.

10. The surgical system of claim 9, wherein the x-ray source and the x-ray detector remain stationary relative to the frame while obtaining scout imaging data.

11. The surgical system of claim 9, wherein rotation of the rotor about the axis moves the x-ray source and the x-ray detector relative to the frame while obtaining three-dimensional imaging data.

12. The surgical system of claim 1, wherein the localizer of the navigation system is configured to optically track states of the plurality of trackers.

13. The surgical system of claim 12, wherein the localizer of the navigation system monitors tracked states of the gantry tracker occurring over the scan period based on optical movement of the gantry tracker occurring during initial movement of the gantry from the first gantry position, during movement interruption of the gantry at the second gantry position, and during intermittent movement of the gantry between the first gantry position and the second gantry position.

14. The surgical system of claim 1, wherein the imaging device further includes a motor to translate the gantry relative to the base.

15. The surgical system of claim 1, wherein the plurality of trackers further includes a patient tracker adapted for attachment relative to the target site.

16. The surgical system of claim 1, further comprising a tabletop support and a fiducial plate connected with the tabletop support, the fiducial plate including a plurality of radio opaque markers arranged in a specific geometry arrangement, the plurality of radio opaque markers configured to appear in the imaging data with the specific geometry arrangement.

17. A surgical system comprising:
an imaging device including:
a base,
a gantry supported for translation relative to the base between a first gantry position and a second gantry position to obtain imaging data of a target site of a patient over a scan period, and
a position sensor to generate gantry data representing a position of the gantry relative to the base between the first gantry position and the second gantry position;
a plurality of trackers a patient tracker adapted for attachment relative to the target site, and a gantry tracker operatively attached to the gantry;
a navigation system including a localizer to track states of the plurality of trackers in a navigation reference frame; and
a controller operatively connected to the imaging device and to the navigation system, the controller being configured to register the imaging data to the navigation reference frame by synchronizing the gantry data generated by the position sensor with changes in tracked states of the gantry tracker occurring over the scan period representing movement of the gantry between the first gantry position and the second gantry position.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. A surgical system comprising:
an imaging device including:
a base,
a tabletop support connected with the base and configured to support a patient;
a gantry supported for translation relative to the base and the tabletop support between a first gantry position and a second gantry position to obtain imaging data of a target site of a patient over a scan period, and
a position sensor to generate gantry data representing a position of the gantry relative to the base and the tabletop support between the first gantry position and the second gantry position;
a plurality of trackers including a gantry tracker operatively attached to the gantry;
a fiducial plate connected with the tabletop support, the fiducial plate including a plurality of radio opaque markers arranged in a specific geometry arrangement, the plurality of radio opaque markers configured to appear in the imaging data with the specific geometry arrangement;
a navigation system including a localizer to track states of the plurality of trackers in a navigation reference frame; and
a controller operatively connected to the imaging device and to the navigation system, the controller being configured to register the imaging data to the navigation reference frame by synchronizing the specific geometry arrangement of the plurality of radio opaque markers in the imaging data with changes in tracked states of the gantry tracker occurring over a scan period representing movement of the gantry between the first gantry position and the second gantry position and the gantry data generated by the position sensor.

31. The surgical system of claim 16, wherein the controller is further configured to synchronize the specific geometry arrangement of the plurality of radio opaque markers in the imaging data with changes in tracked states of the gantry tracker occurring over the scan period representing movement of the gantry between the first gantry position and the second gantry position and the gantry data generated by the position sensor.
